(19) United States
(12) Patent Application Publication
(10) Pub. No.: US 2017/0325869 A1
(43) Pub. Date: Nov. 16, 2017

(54) METHODS OF ABLATING TISSUE

(71) Applicant: COVIDIEN LP, Mansfield, MA (US)

(72) Inventor: JOSEPH D. BRANNAN, LYONS, CO (US)

(21) Appl. No.: 15/150,625

(22) Filed: May 10, 2016

(51) Int. Cl. A61B 18/02; A61B 17/88; A61B 18/18; A61B 18/00

(52) U.S. Cl. CPC A61B 18/02; A61B 18/1815; A61B 17/8805; A61B 2018/00565; A61B 2018/00726; A61B 2018/00994; A61B 2018/00714

(57) ABSTRACT

A method of treating tissue includes positioning a probe in proximity to tissue, reducing a temperature of the tissue such that a temperature of a portion of the tissue that is closer to the probe is less than a temperature of a portion of the tissue that is farther from the probe, and raising a temperature of the tissue such that the temperature of the portion of the tissue that is closer to the probe increases at a faster rate than the temperature of the portion of the tissue farther from the probe.

### BACKGROUND

### 1. Technical Field

The present disclosure relates to methods of ablating tissue and, more specifically, to methods of producing a uniform temperature field within tissue being ablated.

### 2. Discussion of Related Art

Electromagnetic fields can be used to heat and destroy tumor cells. Treatment may involve inserting ablation probes into tissues where cancerous tumors have been identified. Once the ablation probes are properly positioned, the ablation probes induce electromagnetic fields within the tissue surrounding the ablation probes to destroy the cancerous tumors.

In the treatment of diseases such as cancer, certain types of tumor cells have been found to denature at elevated temperatures. In certain tissues (e.g., bone), known treatment methods include heating diseased cells to temperatures of approximately 60° C. to kill the diseased cells. During such heating, the structural integrity of the treated bone can be impacted, requiring the implantation of bone screws and/or the application of bone cement to provide support to the affected area and assist in bone healing.

As an alternative to the above-noted method, cryoablation may be used to destroy tumor cells. Typically, a cryoablation procedure includes freezing the tumor cells to destroy or kill the tumor cells. When cryoablation is used to kill cancerous cells in tissue (e.g., bone), the structure of the bone is not negatively affected by the low temperature as is typically the case with treatments involving heat. However, when a substantial amount of cancerous tissue is removed from the bone, the cavities that remain following such removal may result in a compromised bone structure.

Typically, bone cement would be applied to the subject bone to fill in the cavities and/or repair bone lesions caused by the cancer. However, applying bone cement while the bone is in a frozen state from the cryoablation procedure may be ineffective due to the bone cement not hardening fast enough. Thus, an additional procedure following thawing of the bone may be required to repair bone lesions and/or to fill in the cavities that remain following the removal of the cancerous cells.

Additionally, when ablating diseased or cancerous tissue (e.g., liver, kidney, bone, etc.) using a treatment device that heats tissue (e.g., a microwave antenna), the tissue closer to the device will heat up faster than tissue further from the device, potentially resulting in an unwanted burning of tissue and sometimes damage to healthy tissue adjacent the tissue intended to be treated.

A procedure that generates a uniform temperature field in tissue being treated will result in less damage to healthy tissue that is adjacent to the tissue intended to be treated. In addition, a single procedure that reliably kills diseased cells in tissue without leaving the surrounding tissue frozen, allows for cement to be applied during the procedure.

### SUMMARY

This disclosure relates generally to methods for producing a uniform temperature field within tissue to kill diseased cells (e.g., cancerous cells) within the tissue. The method generally includes cooling the cancerous cells (e.g., a tumor) and then delivering RF energy (e.g., microwave energy) to the cooled cells to heat and destroy the tumor. In embodiments, the cooling process may destroy the tumor. When the method is used to treat bone, the method may include applying cement to the bone after the diseased cells are killed to reinforce the bone.

In an aspect of the present disclosure, a method of treating tissue is provided and includes positioning a probe in proximity to tissue; reducing a temperature of the tissue such that a temperature of a portion of the tissue that is closer to the probe is less than a temperature of a portion of the tissue that is farther from the probe; and raising a temperature of the tissue such that the temperature of the portion of the tissue that is closer to the probe increases at a faster rate than the temperature of the portion of the tissue farther from the probe.

In embodiments, the temperature of the tissue may be raised until the first and second portions of the tissue achieve substantially the same temperature. The temperature of the first and second portions of the tissue may be raised to a temperature that destroys the tissue. The temperature that destroys the tissue may be 40° C. to 100° C. The temperature that destroys the tissue may be 60° C.

In embodiments, reducing the temperature of the tissue may include releasing cryogen from a cryogenic source. Raising the temperature of the tissue may include radiating microwave energy into the tissue while the probe is in the same location as when the probe reduces the temperature of the tissue.

In embodiments, the probe may radiate microwave energy into the tissue after a temperature of the probe is reduced to below 0° C. The portion of the tissue closer to the probe may be frozen and the portion of the tissue farther from the probe may not be frozen.

In embodiments, the method may include alternating, in a cyclical manner, radiation of microwave energy from the probe and releasing cryogen into the probe.

In embodiments, the probe may be a cryoprobe, and the method may further include moving the cryoprobe away from the tissue after reducing the temperature of the tissue; and positioning a microwave ablation probe in proximity to tissue, wherein the temperature of the tissue is raised using the microwave ablation probe.

In embodiments, the temperature of the tissue may be raised after being reduced.

In embodiments, the method may further include applying cement to the tissue.

In another aspect of the present disclosure, a method of treating tissue is provided and includes positioning a probe in proximity to tissue; reducing a temperature of the probe, thereby reducing a temperature of a proximal area of the tissue to a first temperature and reducing a temperature of a distal area of the tissue to a second temperature, greater than the first temperature; and delivering radiation to the tissue to raise the temperature of the proximal and distal areas of the tissue to a third temperature, greater than the second temperature.

In embodiments, the proximal and distal areas may achieve the third temperature at substantially the same time.

In embodiments, the radiation may be emitted from the probe while the probe is in the same location as when the temperature of the probe is reduced.

In embodiments, the method may further include alternating, in a cyclical manner, radiation of microwave energy from the probe and releasing cryogen into the probe.

In embodiments, the probe may be a cryoprobe, and the method may further include moving the cryoprobe away from the tissue after reducing the temperature of the proximal and distal areas to the respective first and second temperatures; and positioning a microwave ablation probe adjacent the proximal area, wherein the temperature of the proximal and distal areas is raised using the microwave ablation probe.

In yet another aspect of the present disclosure, a method of treating tissue is provided and includes positioning a probe in proximity to a proximal area of tissue; reducing a temperature of the probe to reduce a temperature of the proximal area of the tissue to a first temperature and reducing a temperature of a distal area of the tissue to a second temperature, greater than the first temperature, the distal area of the tissue being further from the probe than the proximal area of the tissue; and delivering radiation into the tissue to raise the temperature of the proximal and distal areas of the tissue to a third temperature, greater than the second temperature, wherein the third temperature effects ablation of the proximal and distal areas of the tissue.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a schematic illustration of a microwave ablation system used for treating tissue in accordance with the methods disclosed herein;

FIG. 2 is a side view of a cryoablation system used for treating tissue in accordance with the methods disclosed herein;

FIG. 3A is a plan view of a microwave ablation probe of the system of FIG. 1 disposed adjacent tissue to be treated;

FIG. 3B is a plan view of a cryoprobe of the system of FIG. 2 disposed adjacent the tissue to be treated; and

FIG. 4 is a plan view of a dual-functioning probe disposed adjacent tissue to be treated.

### DETAILED DESCRIPTION

Embodiments of the disclosed ablation systems, probes, and methods of use are described with reference to the accompanying drawings. Like reference numerals may refer to similar or identical elements throughout the description of the figures. As shown in the drawings and as used in this description, the term “proximal” refers to that portion of the described probe, or tissue area, that is closer to the user of the probe, and the term “distal” refers to that portion of the probe, or tissue area, that is farther from the user of the probe.

As it is used in this description, “microwave” generally refers to electromagnetic waves in the frequency range of 300 megahertz (MHz) (3×10⁸ cycles/second) to 300 gigahertz (GHz) (3×10¹¹ cycles/second). As it is used in this description, “ablation procedure” generally refers to any ablation procedure, such as, for example, microwave ablation, radiofrequency (RF) ablation, or cryoablation. As it is used in this description, “fluid” generally refers to a liquid, a gas, or both. The term “coolant” may be used interchangeably with the term “fluid.”

Reference will now be made in detail to embodiments of the present disclosure. While certain exemplary embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the scope of the embodiments of the present disclosure as defined by the appended claims.

The present disclosure provides methods of treating tissue by applying a cryoprobe to tissue to reduce the temperature of a portion of the tissue that is closer to the cryoprobe a greater amount than a portion of the tissue that is further from the cryoprobe. Radiation is also applied to the tissue to raise the temperature of both portions of the tissue to the same temperature, wherein the temperature is known to destroy tissue.

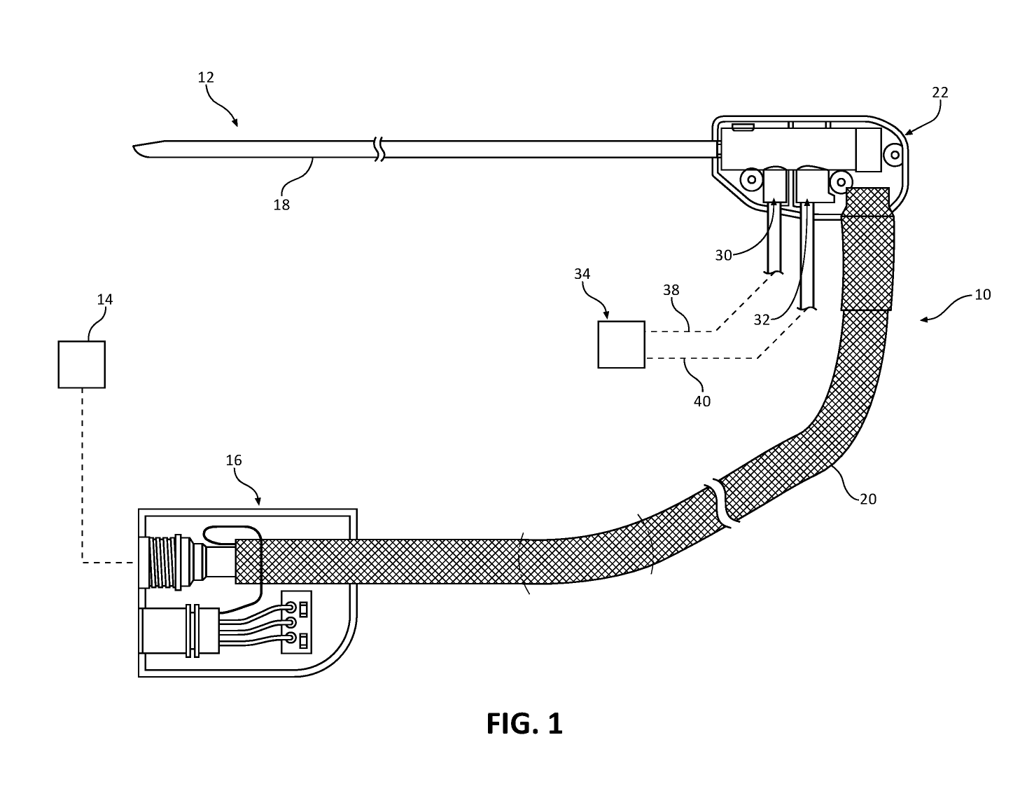

With reference to FIG. 1, a microwave ablation system 10 is provided for use with the methods of treating tissue described in detail below. Microwave ablation system 10 generally includes a microwave ablation probe 12, a microwave generator 14, and a coolant supply system 34. Generator 14 is configured to provide microwave energy at an operational frequency from about 500 MHz to about 5000 MHz, although other suitable frequencies are also contemplated. In some embodiments, generator 14 may generate any suitable type of energy, for example, RF energy, or ultrasonic energy.

Probe 12 and generator 14 are coupled to one another via a connector assembly 16 and a cable assembly 20. Connector assembly 16 is a cable connector suitable to operably connect cable assembly 20 to generator 14. Connector assembly 16 may house a memory (e.g., an EEPROM) storing a variety of information regarding various components of system 10. For example, the memory may store identification information that can be used by generator 14 to determine the identification of probes connected to generator 14. Based on the determined identification of a probe, generator 14 may or may not provide energy to the probe. For example, if the identification information stored in memory does not match identification information provided by the probe (e.g., via a RFID tag on the probe), generator 14 will not provide energy to the connected probe.

Cable assembly 20 interconnects connector assembly 16 and probe 12 to allow for the transfer of energy from generator 14 to probe 12. Cable assembly 20 may be any suitable, flexible transmission line, such as a coaxial cable, including an inner conductor, a dielectric material coaxially surrounding the inner conductor, and an outer conductor coaxially surrounding the dielectric material. Cable assembly 20 may be provided with an outer coating or sleeve disposed about the outer conductor. The sleeve may be formed of any suitable insulative material, and may be applied by any suitable method, e.g., heat shrinking, over-molding, coating, spraying, dipping, powder coating, and/or film deposition.

Probe 12 includes a radiating portion 18 that radiates energy provided by generator 14. Radiating portion 18 is coupled to cable assembly 20 through a handle assembly 22. Handle assembly 22 has an outlet fluid port 30 and an inlet fluid port 32 each in fluid communication with an interior chamber (not explicitly shown) defined in probe 12. Thus, coolant fluid may circulate from ports 30 and 32 around the interior chamber or chambers of probe 12 to cool probe 12 during use.

Ports 30 and 32 are coupled to a coolant supply system 34 via supply lines 38, 40, respectively. Coolant supply system 34 includes a supply tank (not explicitly shown) and a supply pump (not explicitly shown). The supply pump may be a peristaltic pump or any other suitable pump configured to circulate coolant fluid from the supply tank and into probe 12. The supply tank stores the coolant fluid and, in one embodiment, may maintain the fluid at a predetermined temperature. More specifically, the supply tank may include a coolant unit that cools the returning liquid from the probe 12. In another embodiment, the coolant fluid may be a gas and/or a mixture of fluid and gas.

For a more detailed description of a microwave ablation system, reference may be made to U.S. Patent Application Publication No. 2014/0276033, filed on Mar. 15, 2013, the entire contents of which are incorporated by reference herein.

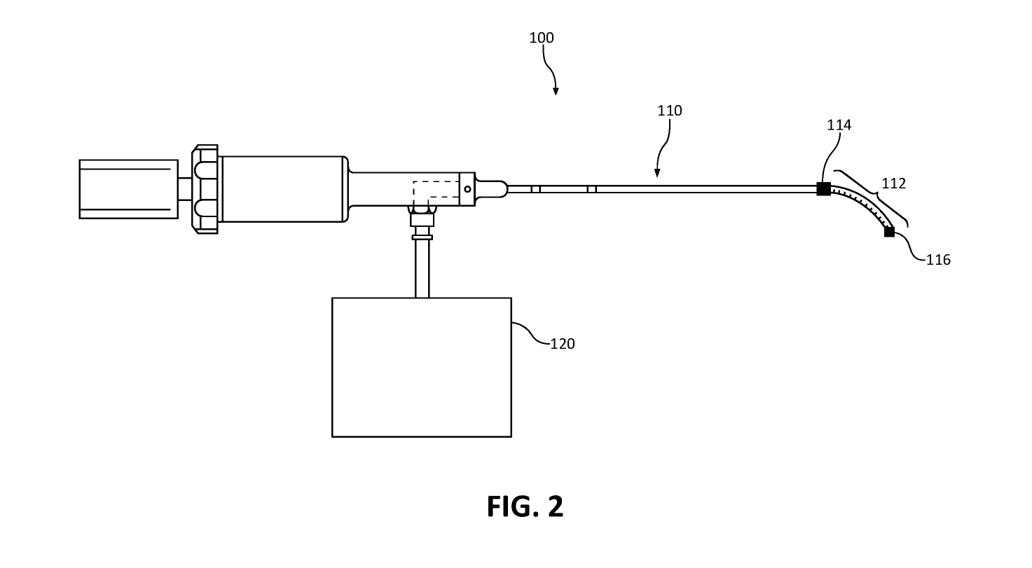

With reference to FIG. 2, a cryoablation system 100 is provided for use with the methods of treating tissue described in detail below. Cryoablation system 100 includes a cryoprobe 110 and a cryogenic source 120. Cryoprobe 110 is configured to reduce the temperature of tissue to a threshold temperature at which the subject tissue freezes. For example, in one embodiment, the cryogenic source 120 may be used to reduce a temperature of a distal portion 112 of cryoprobe 110. Distal portion 112 of cryoprobe 110 includes longitudinally-spaced cryotips 114, 116 configured as energy delivery elements. Cryogen from cryogenic source 120 causes one or more of cryotips 114, 116 to apply extreme cold to tissue to be ablated. In embodiments, distal portion 112 of cryoprobe 110 may be steerable and bendable.

A variety of substances may be used for cryogen. Examples of such substances include, but are not limited to, liquid nitrogen, helium, argon, hydrogen, oxygen, and the like.

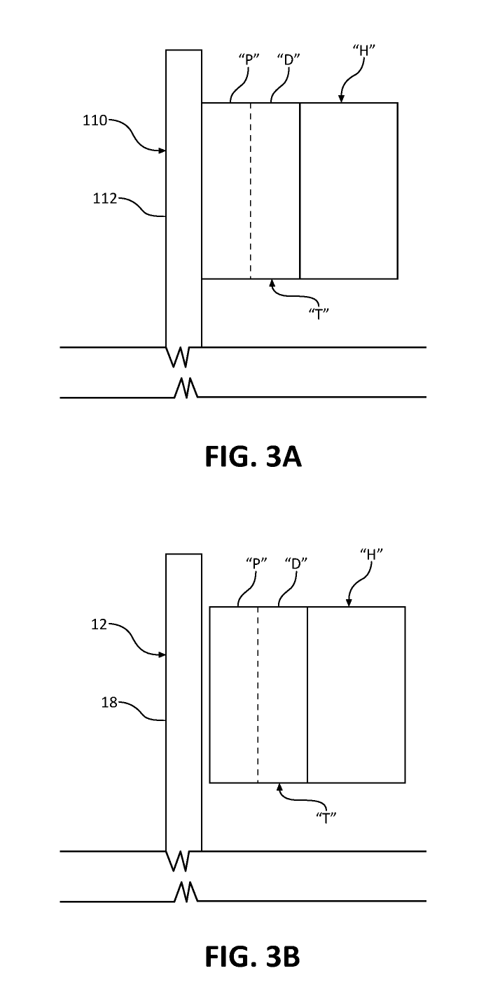

With reference to FIGS. 3A and 3B, a method of treating cancerous tissue or a tumor “T” is provided, which utilizes the microwave ablation system 10 described above with reference to FIG. 1 and the cryoablation system 100 described above with reference to FIG. 2 that are used interchangeably by being passed in and out of an access cannula. The method uses microwave ablation probe 12 and cryoprobe 110 to ablate tumor “T” by generating a uniform temperature field throughout the tumor “T.” In particular, a minimally invasive procedure, for example, a laparoscopic procedure is used to gain access to a surgical site within a patient. A cannula, or any suitable access port, having a stylet at its distal end may be used to access a body cavity of a patient. Cryoprobe 110 is inserted within the cannula (not shown) that extends into the surgical site. Distal portion 112 of cryoprobe 110 is disposed in suitable proximity to the tumor “T,” for example, next to or in contact therewith. The tumor “T” has a proximal area “P” and a distal area “D,” which is located further away from cryoprobe 110 than proximal area “P.” Healthy tissue “H” (e.g., liver, kidney, bone, etc.) surrounds the tumor “T.”

With cryoprobe 110 disposed in suitable proximity to proximal area “P” of the tumor “T,” cryogenic source 120 (FIG. 2) provides cryogen to cryoprobe 110 to reduce the temperature of cryoprobe 110, and, in turn, reducing the temperature of proximal area “P.” Continued activation of cryoprobe 110 for a threshold amount of time eventually reduces the temperature of distal area “D.” The threshold amount of time is approximately the time required for heat to transfer the distance from distal area “D” to cryoprobe 110.

Cryoprobe 110 is held in suitable proximity to proximal area “P” in an activated state until proximal area “P” is reduced to a first temperature, for example, 0° C. or below, and distal area “D” is reduced to a second temperature that is below the first temperature of proximal area “P,” for example, between 0° C. and 37° C. (body temperature). In some embodiments, the proximal area “P” may be reduced to a temperature that is above 0° C. In some embodiments, the distal area “D” may be reduced to a temperature that is below 0° C.

Due to proximal area “P” being closer to cryoprobe 110 than distal area “D,” proximal area “P” will achieve a lower temperature than distal area “D.” As such, the temperature of the tumor “T” is lowest at the proximal-most surface of proximal area “P,” and the temperature of the tumor “T” is highest at the distal-most surface of distal area “D.” The temperature of the tumor “T” decreases at a faster rate when moving in a proximal direction between proximal area “P” and distal area “D.” However, as can be appreciated, the temperature of each of proximal area “P” and distal area “D” may approximate the temperature of cryoprobe 110 following a prolonged activation of cryoprobe 110.

Upon cooling proximal area “P” and distal area “D” of the tumor “T” to the respective first and second temperatures, cryoprobe 110 is passed through the cannula and removed from the surgical site. Microwave ablation probe 12 is inserted within the cannula such that radiating portion 18 is disposed in suitable proximity to proximal area “P” of the tumor “T.” With radiating portion 18 of microwave ablation probe 12 disposed in contact with proximal area “P,” microwave ablation probe 12 is activated to radiate microwave radiation into the proximal and distal areas “P,” “D” of the tumor “T.” In some embodiments, other types of radiofrequency energy (e.g., RF, ultrasonic, or the like) may be emitted from microwave ablation probe 12 to heat the tumor “T.”

The delivery of microwave radiation into the tumor “T” raises the temperature of the proximal and distal areas “P,” “D” of the tumor “T” above the respective first and second temperatures. Activation of microwave ablation probe 12 is continued until the temperature of each of the proximal and distal areas “P,” “D” of the tumor “T” is raised from the respective first and second temperatures to a threshold temperature at which the proximal and distal areas “P,” “D” of the tumor “T” are ablated. In this way, a uniform temperature field is generated throughout the tumor “T” at approximately the same point in time (i.e., the entire tumor “T” is substantially the same temperature). The threshold temperature may be between about 40° C. and 100° C. In some embodiments, the threshold temperature may be about 60° C.

Proximal area “P” absorbs more microwave energy from microwave ablation probe 12 than distal area 110 because of the proximity of proximal area “P” to microwave ablation probe 12. As such, the temperature of proximal area “P” is increased from its first temperature to the threshold temperature at a faster rate than the rate at which the temperature of the distal area “D” is increased from its second temperature to the threshold temperature. The faster rate at which proximal area “P” is heated to the threshold temperature compared to distal area “D” is balanced by proximal area “P” being at a lower starting temperature than distal area “D.” In this way, even though proximal area “P” starts at a lower temperature than distal area “D,” proximal area “P” is heated to the threshold temperature (e.g., about 60° C.) in approximately the same amount of time as distal area “D.”

Without first generating a lower temperature in proximal area “P” compared to distal area “D,” proximal area “P” may be overheated during ablation of tumor “T,” which may result in charring of the proximal area “P.” Alternatively, distal area “D” may be under-heated during ablation of tumor “T” resulting in some of the cells of tumor “T” located further from the microwave ablation probe 12 surviving the ablation procedure.

It is contemplated that one or more visualization techniques including ultrasound, computed tomography (CT), fluoroscopy, and direct visualization may be used to accurately guide the probes 12, 110 into the area of tissue to be treated.

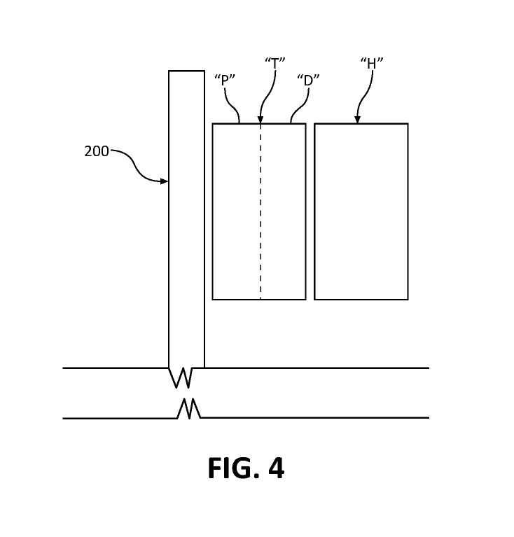

With reference to FIG. 4, another method of ablation a tumor “T” by generating a uniform temperature field throughout the tumor “T.” The method with reference to FIG. 4 is similar to the method described above with reference to FIGS. 3A and 3B. Thus, to prevent unnecessary repetition, only selected differences between the methods are described.

The method utilizes a probe 200 having the functionality of both the microwave ablation probe 12 described above with reference to FIG. 1 and the cryoprobe 110 described above with reference to FIG. 2. A cannula, or any suitable access port, having a stylet at its distal end may be used to access a body cavity of a patient. Probe 200 is inserted through the cannula into the surgical site and disposed in suitable proximity to the tumor “T.” The tumor “T” has a proximal area “P” and a distal area “D,” which is located further away from probe than proximal area. Healthy tissue “H” (e.g., liver, kidney, bone, etc.) surrounds the tumor “T.”

With probe 200 disposed in suitable proximity to proximal area “P” of the tumor “T,” a cryogen source (not shown) coupled to probe 200 provides cryogen to probe 200 to reduce the temperature of probe 200, thereby reducing the temperature of proximal area “P.” Continued activation of the cooling function of probe 200 for a threshold amount of time eventually reduces the temperature of distal area “D.” The threshold amount of time is approximately the time required for heat to transfer the distance from distal area “D” to probe 200.

Probe 200 is held in suitable proximity to proximal area “P” in an activated state until proximal area “P” is reduced to a first temperature (e.g., 0° C. or below) and distal area “D” is reduced to a second temperature that is below the first temperature of proximal area “P,” for example, between 0° C. and 37° C. (body temperature). In some embodiments, the proximal area “P” may be reduced to a temperature that is above 0° C. In some embodiments, the distal area “D” may be reduced to a temperature that is below 0° C.

Upon cooling proximal area “P” and distal area “D” of the tumor “T” to the respective first and second temperatures, the microwave radiation function of probe 200 is activated to radiate microwave radiation into the proximal and distal areas “P,” “D” of the tumor “T.” In some embodiments, probe 200 may be configured to emit any type of radiofrequency energy to heat the tumor “T.”

The delivery of microwave radiation into the tumor “T” raises the temperature of the proximal and distal areas “P,” “D” of the tumor “T” above the respective first and second temperatures. Activation of the microwave ablation functionality of probe 200 is continued until the temperature of the proximal and distal areas “P,” “D” of the tumor “T” is raised from the respective first and second temperatures to a threshold temperature at which the proximal and distal areas “P,” “D” of the tumor “T” are ablated. In this way, a uniform temperature field is generated throughout the tumor “T” at approximately the same point in time such that the entire tumor “T” is substantially the same temperature. The threshold temperature is between about 40° C. and 100° C. In some embodiments, the threshold temperature may be about 60° C. In embodiments, probe 200 may be activated to heat the tumor “T” to a temperature below the threshold temperature.

In embodiments, probe 200 may be cycled, in a pulsating manner, between activation of the microwave ablation and cryoablation functionalities to cyclically heat and cool the tumor “T.” During each cycle, proximal and distal areas “P,” “D” are reduced to a temperature of or below 0° C. to freeze the tumor “T,” and then raised to a temperature above 0° C. to unfreeze the tumor “T.” In some embodiments, this cycling between microwave ablation and cryoablation may continue until proximal and distal areas “P,” “D” both achieve the threshold temperature.

In embodiments, when the subject tissue is bone, bone cement may be applied to the bone to fill any cavities that remain following completion of any of the above-described ablation procedures. A tool used to deliver the bone cement may be passed through an access cannula into the surgical site. The bone cement is more effective when the subject bone tissue is at the threshold temperature rather than at a temperature around or below 0° C.

It is envisioned that each of the disclosed methods of treating tissue may be performed under CT, MRI, direct thermometry using MRI or CT, or ultrasound for conformational density measurements. Heat or ice build-up may be used as visual cues to determine when to switch between the cooling portion of the ablation procedure and the heating portion of the ablation procedure.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A method of treating tissue, comprising:
positioning a probe in proximity to tissue;
reducing a temperature of the tissue such that a temperature of a portion of the tissue that is closer to the probe is less than a temperature of a portion of the tissue that is farther from the probe; and
raising a temperature of the tissue such that the temperature of the portion of the tissue that is closer to the probe increases at a faster rate than the temperature of the portion of the tissue farther from the probe.

2. The method according to claim 1, wherein the temperature of the tissue is raised until the first and second portions of the tissue achieve substantially the same temperature.

3. The method according to claim 2, wherein the temperature of the first and second portions of the tissue is raised to a temperature that destroys the tissue.

4. The method according to claim 3, wherein the temperature that destroys the tissue is 40° C. to 100° C.

5. The method according to claim 4, wherein the temperature that destroys the tissue is 60° C.

6. The method according to claim 1, wherein reducing the temperature of the tissue includes releasing cryogen from a cryogenic source.

7. The method according to claim 6, wherein raising the temperature of the tissue includes radiating microwave energy into the tissue while the probe is in the same location as when the probe reduces the temperature of the tissue.

8. The method according to claim 1, wherein the probe radiates microwave energy into the tissue after a temperature of the probe is reduced to below 0° C.

9. The method according to claim 8, wherein the portion of the tissue closer to the probe is frozen and the portion of the tissue farther from the probe is not frozen.

10. The method according to claim 1, further comprising alternating, in a cyclical manner, radiation of microwave energy from the probe and releasing cryogen into the probe.

11. The method according to claim 1, wherein the probe is a cryoprobe, the method further comprising:
moving the cryoprobe away from the tissue after reducing the temperature of the tissue; and
positioning a microwave ablation probe in suitable proximity to tissue, wherein the temperature of the tissue is raised using the microwave ablation probe.

12. The method according to claim 1, wherein the temperature of the tissue is raised after being reduced.

13. The method according to claim 1, further comprising applying cement to the tissue.

14. A method of treating tissue, comprising:
positioning a probe in proximity to tissue;
reducing a temperature of the probe, thereby reducing a temperature of a proximal area of the tissue to a first temperature and reducing a temperature of a distal area of the tissue to a second temperature, greater than the first temperature; and
delivering radiation to the tissue to raise the temperature of the proximal and distal areas of the tissue to a third temperature, greater than the second temperature.

15. The method according to claim 14, wherein the proximal and distal areas achieve the third temperature at substantially the same time.

16. The method according to claim 14, wherein the radiation is emitted from the probe while the probe is in the same location as when the temperature of the probe is reduced.

17. The method according to claim 14, further comprising alternating, in a cyclical manner, radiation of microwave energy from the probe and releasing cryogen into the probe.

18. The method according to claim 14, wherein the probe is a cryoprobe, the method further comprising:
moving the cryoprobe away from the tissue after reducing the temperature of the proximal and distal areas to the respective first and second temperatures; and
positioning a microwave ablation probe adjacent the proximal area, wherein the temperature of the proximal and distal areas is raised using the microwave ablation probe.

19. A method of treating tissue, comprising:
positioning a probe in proximity to a proximal area of tissue;
reducing a temperature of the probe to reduce a temperature of the proximal area of the tissue to a first temperature and reducing a temperature of a distal area of the tissue to a second temperature, greater than the first temperature, the distal area of the tissue being further from the probe than the proximal area of the tissue; and
delivering radiation into the tissue to raise the temperature of the proximal and distal areas of the tissue to a third temperature, greater than the second temperature, wherein the third temperature effects ablation of the proximal and distal areas of the tissue.
